(19) United States
(12) Patent Application Publication
(10) Pub. No.: US 2004/0132070 A1
(43) Pub. Date: Jul. 8, 2004

(54) Nonotube-based electronic detection of biological molecules

(75) Inventors: Alexander Star, Albany, CA (US); George Gruner, Los Angeles, CA (US)

Correspondence Address: O'MELVENY & MYERS LLP, 400 South Hope Street, Los Angeles, CA, 90071-2899, US

(73) Assignee: NANOMIX, INC.

(21) Appl. No.: 10/704,066

(22) Filed: Nov. 7, 2003

Related U.S. Application Data
(63) Continuation-in-part of application No. 10/656,898, filed on Sep. 5, 2003.
(63) Continuation-in-part of application No. 10/345,783, filed on Jan. 16, 2003.
(60) Provisional application No. 60/424,892, filed on Nov. 8, 2002.
(60) Provisional application No. 60/408,547, filed on Sep. 5, 2002.
(60) Provisional application No. 60/349,670, filed on Jan. 16, 2002.

(51) Int. Cl. C12Q 1/68

(52) U.S. Cl. 435/6

(57) ABSTRACT

Nanoscale field effect transistor devices with carbon nanotubes as the conducting channel are used to detect protein-protein binding. A coating of an electron-donating polymer is applied to a nanotube device, and a receptor compound is bound to the polymer. The receptor compound is configured to bind a specific biological molecule or molecules. The device coated with the polymer coating and receptor compound may be operated as a p-type field-effect transducer. For example, upon exposure to biological molecules bound by the receptor, the conductance at negative voltage may be markedly reduced, thereby establishing an electronic signal response.

### CROSS-REFERENCE TO RELATED APPLICATIONS

This application claims priority pursuant to 35 U.S.C. § 119(e) to U.S. Provisional Application No. 60/424,892, filed Nov. 8, 2002. This application is a continuation-in-part of co-pending application Ser. No. 10/656,898, filed Sep. 5, 2003, which claims priority to Provisional Application No. 60/408,547, filed Sep. 5, 2002. This application is also a continuation-in-part of co-pending application Ser. No. 10/345,783, filed Jan. 16, 2003, which claims priority to Provisional Application No. 60/349,670, filed Jan. 16, 2002. Each of the foregoing provisional and non-provisional applications are specifically incorporated herein, in their entirety, by reference.

### BACKGROUND OF THE INVENTION

1. Field of the Invention

The present invention relates to the detection of biological molecules by nanotube-based sensors.

2. Description of Related Art

Current biological sensing techniques commonly rely on optical detection principles that are inherently complex, require multiple steps between the actual engagement of the analyte and the generation of a signal, multiple reagents, preparative steps, signal amplification, complex data analysis and/or relatively large sample size.

Nanowires and nanotubes, by virtue of their small size, large surface area, and near one-dimensionality of electronic transport, are promising candidates for electronic detection of chemical and biological species (1). Field effect transistors (FETs) fabricated from component semiconducting single wall carbon nanotubes (NTs) have been studied extensively for their potential as sensors. A number of properties of these devices have been identified, and different mechanisms have been proposed to describe their sensing behavior. Devices that incorporate carbon nanotubes have been found to be sensitive to various gases, such as oxygen and ammonia, and these observations have confirmed the notion that such devices can operate as sensitive chemical sensors.

Single-walled nanotube (“SWNT”) devices, including field-effect transistors (“FET's”) and resistors, can be fabricated using nanotubes grown on silicon or other substrates by chemical vapor deposition from iron-containing catalyst nanoparticles with methane/hydrogen gas mixture at 900° C. Other catalyst materials and gas mixtures can be used to grow nanotubes on substrates, and other electrode materials and nanostructure configurations and have been described previously by Gabriel et al. in U.S. patent application Ser. No. 10/099,664 and in U.S. patent application Ser. No. 10/177,929, both of which are incorporated by reference herein. Currently, technology for constructing practical nanostructure devices is in its infancy. While nanotube structures show promise for use as sensor devices and transistors, current technology is limited in many ways.

For example, it is desirable to take advantage of the small size and sensitivity of nanotube and other nanostructure sensors to sense biological molecules, such as proteins. But a useful sensor of this type should selectively and reliably respond to a molecular target of a specific type. For example, it may be desirable to selectively sense a specific protein, while not responding to the presence of other proteins in the sample. Examples of covalent chemical attachment of biological molecules to nanotubes, including proteins and DNA, are known in the art, although it has not been convincingly demonstrated that useful detection of specific proteins or other large biomolecules can be accomplished in this way. For one thing, covalent chemical attachment has the disadvantage of impairing physical properties of carbon nanotubes, making structures of this type less useful as practical sensors. In addition, the carbon nanotubes are hydrophobic, and generally non-selective in reacting with biomolecules.

It is desirable, therefore, to provide a nanotube sensing device that is biocompatible, and exhibits a high degree of selectivity to particular biomolecular targets.

### SUMMARY OF THE INVENTION

In accordance with embodiments of the present invention, a nanotube sensor architecture is provided, which allows the detection of protein-protein interactions and, at the same time, reduces or eliminates non-specific binding. The sensor may be operated as a nanostructure field effect transistor, to detect the presence of a specific protein or other biomolecule. Further provided are methods for making and operating the sensing device.

A nanostructure device according to the invention may comprise a nanotube, such as a carbon nanotube, disposed along a substrate, such as a silicon substrate. The nanotube may span two conductive elements, which may serve as electrical terminals, or as a source and drain. A passivation layer, such as of silicon monoxide, may be deposited over the conductive elements and a portion of the nanotube, leaving a portion of the nanotube between the conductive elements exposed. The nanotube may be coated with a thin polymer layer, for example comprising poly(ethylene imine) (“PEI”) and poly(ethylene glycol) (PEG). In this configuration, the device may be operated as an n-type FET, as further described in application Ser. No. 10/656,898. Advantageously, the polymer layer is hydrophilic and biocompatible, making the nanotube device essentially non-reactive to large biomolecules such as proteins.

A bioreceptor layer may be attached over the polymer layer, configured for reactivity to a specific biomolecule. For example, biotin is known to selectively bind to streptavidin. The bioreceptor layer should be configured to bind to the polymer layer. For example, a solution of biotin-N-hydroxysuccinimide ester reacts with primary amines in PEI, thereby binding biotin molecules to the polymer layer. The bioreceptor layer may comprise a mono-molecular layer, comprised of discrete bioreceptor molecules attached to the polymer layer.

The resulting device will exhibit transconductance that varies depending on the presence of the targeted biomolecule in its sample environment. For example, a bioreceptor layer comprised of attached biotin molecules will selectively bind to streptavidin, causing a measurable decrease in transconductance at negative gate voltages. The device may therefore be used as a sensor for streptavidin. To sense other biomolecules, the device may be provided with a different bioreceptor layer that is configured to bind to the desired target.

A more complete understanding of the biomolecular sensor will be afforded to those skilled in the art, as well as a realization of additional advantages and objects thereof, by a consideration of the following detailed description of the preferred embodiment. Reference will be made to the appended sheets of drawings which will first be described briefly.

### BRIEF DESCRIPTION OF THE DRAWINGS

FIG. 1 is a schematic diagram of a nanotube field effect transistor (NTFET) configured as a biomolecule sensor according to the invention.

FIG. 2 is a flow chart showing exemplary steps of a method for making a nanotube biosensor according to the invention.

FIG. 3A is a schematic of a chemical scheme for bonding biotin to a PEI/PEG polymer layer over a nanotube.

FIG. 3B is a chart comparing transconductance of a native PEI/PEG-coated NTFET device with its transconductance after 1 hour, and after 18 hours of being reacted with biotin-N-hydroxysuccinimide ester.

FIG. 4 is a chart comparing transconductance of a bare NTFET to a NTFET coated with a PEI/PEG polymer layer and a NTFET with a biotinylated PEI/PEG layer.

FIG. 5 is a chart comparing the transconductance of a biotinylated, PEI/PEG-coated NTFET device, in the absence and presence of streptavidin.

FIG. 6 is a chart comparing the transconductance of a bare NTFET device, in the absence and presence of streptavidin.

FIG. 7 is a chart comparing the transconductance of a PEI/PEG-coated NTFET device without biotin receptors, in the absence and presence of streptavidin.

FIG. 8 is a chart comparing the transconductance of a biotinylated, PEI/PEG-coated NTFET device, in the absence and presence of streptavidin that has been complexated with biotin, thereby blocking its binding sites.

### DETAILED DESCRIPTION OF THE PREFERRED EMBODIMENT

The present invention provides a nanotube sensor to selectively sense biological molecules, that overcomes the limitations of the prior art. These advancements have been demonstrated by a nanotube sensor according to the invention, which has been shown to be selectively sensitive to the well-characterized ligand-receptor binding of biotin-streptavidin.

In general, the invention provides a sensor architecture that allows the detection of protein-protein interactions, and also reduces or eliminates non-specific binding. An inherently hydrophobic NT-FET, covered with a polymer coating layer with hydrophilic properties, is used as a transducer. The hydrophilicity of the polymer layer reduces the affinity of nanotubes towards non-specific protein binding, which is favored by a hydrophobic environment. In the exemplary embodiment detailed below, biotin is covalently attached to the polymer. When in use, the attached biotin binds with the complementary protein streptavidin, and the formation of the streptavidin-biotin complex is electronically detectable. The streptavidin-biotin complex may serve as a model system for protein interactions, as it has been extensively studied, and the binding is well understood. However, the invention is not limited thereby.

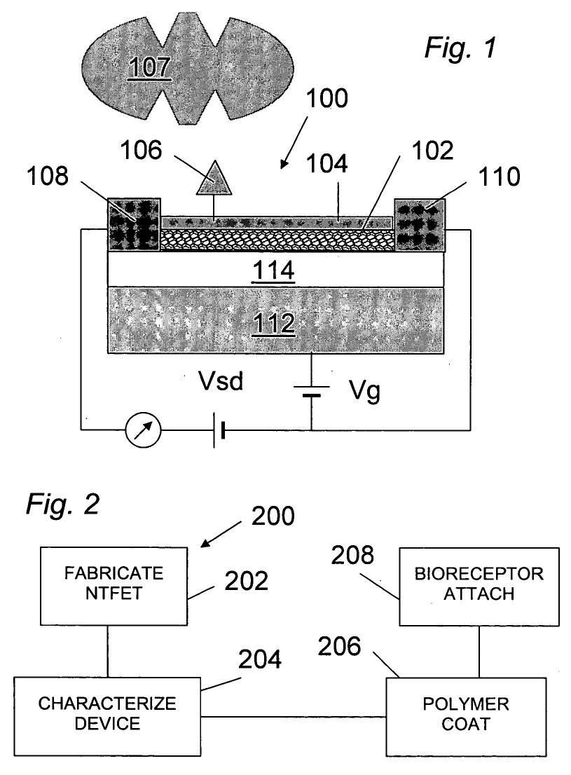

FIG. 1 schematically depicts a sensor 100 that uses a carbon nanotube 102 as a transducer. Nanotube 102 is covered with a polymer coating 104 that has hydrophilic properties and onto which a bioreceptor molecule 106 is attached by a chemical bond to the underlying layer. Bioreceptor 106 may be selected for its selectivity in binding to a biomolecule target 107. Various receptor/target combinations are known, or may be discovered. In an embodiment, the receptor 106 is biotin, and the target 107 is streptavidin. Additional bioreceptor molecules of the same or different types as molecule 106 may additionally be attached to polymer layer 104. A plurality of such bioreceptor molecules (not shown) may disposed over the surface of the polymer layer. The nanotube 102 may be connected to a source electrode 108 and a drain electrode 110 on gate 112. A passivation layer 114 as known in the art, such as SiO₂, may cover the gate substrate 112, which may comprise a silicon or other suitable material.

Functionalization via polymer layer 104 in this sensor architecture has several advantages. First, the polymer is used to attach molecular receptor molecules to the sidewalls of nanotubes, thereby avoiding covalent chemical attachment of biological molecules to nanotubes. Second, polymer coatings have been shown to modify the characteristics of nanotube FET devices, and thus the coating process can be readily monitored. In particular, coating NTFETs with polyethylene imine (PEI) polymer advantageously shifts the device characteristic from p- to n-type. Third, the polymer coating may be useful for preventing nonspecific binding of proteins.

The effect of polymer coating, attachment of a bioreceptor, and subsequent capture of a biomolecule by the bioreceptor on the transconductance of a sensor device according to the invention may be understood as follows, although the invention is not limited thereby. Coating with poly(ethylene imine) (PEI) leads to n-type doping, due to the electron-donating NH₂ groups. PEI is but one example of a polymeric compound that can be utilized in such a way; other examples include poly(ethanol amine) as well as poly(ethylene glycol) (PEG) and polytetrahydrofurane bis(3-aminopropyl)-terminated polymer. Attachment of a bioreceptor, such as biotin, to PEI is through covalent binding to the primary NH₂ group, which would be expected to reduce the overall electron donating function of PEI and cause a transconductance profile that is consistent with indicating removal of electrons from the device. As only the primary NH₂ sites are involved in binding to biotin, the p-type conductance observed before coating is not fully recovered. It is reasonable to postulate that upon streptavidin-biotin binding, geometric changes occur which locally perturb the coating, thereby reducing the effectiveness of the charge transfer and altering the transconductance of the device. It is worth noting that functionalization via the primary NH₂ group of the PEI or other polymer layer could be applied to oligonucleotides, as well as to proteins.

Besides providing desirable electrical properties, layer 104, due to its hydrophilic qualities, may reduce the affinity of nanotubes toward protein binding and thereby improve the selectivity of the device. A variety of polymer coatings and self-assembled mono-molecular layers have been used to prevent binding of undesired species on surfaces for biosensor and biomedical device applications, and may also be suitable for use with the invention. Among the various available polymers for coating, poly(ethylene glycol) is one of the most effective and widely used.

An exemplary method 200 for fabricating FET devices like device 100 with nanotubes as the conducting channel is diagrammed in FIG. 2. At step 202, a p-type NTFET may be fabricated using nanotubes grown by chemical vapor deposition (CVD) on 200 nm of silicon dioxide on doped silicon from iron nanoparticles with methane/hydrogen gas mixture at 900° C. Electrical leads may be patterned on top of the nanotubes from titanium films 35 nm thick capped with gold layers 5 nm thick, with a gap of 0.5 to 0.75 μm between source and drain. Multiple nanotubes may be connected to the source and drain electrodes, with the individual tubes varying from metallic to semiconducting. Consequently, a range of device modulations (expressed as the ratio of the “on” to the “off” source-drain current, measured at −p10 V and +10 V gate voltage, respectively) may be observed. Such devices will display p-type transistor behavior prior to functionalization with a suitable polymer layer. Exemplary devices resulting from the foregoing process may have 0.5 μm wide pairs of electrical leads separated by 0.5 to 0.75 μm gaps, and these gaps may be bridged by 1 to about 5 nanotubes along a 10 μm length of a pair of leads. It should be apparent that numerous other configurations may also be suitable.

At step 204, the device characteristic for the NTFET may be determined. As used herein, “device characteristic” refers to the dependence of the source-drain current, I_{sd}, as function of the gate voltage V_{g}, I_{sd}(V_{g}), measured from +10 V to −10 V. Any other suitable measure may also be used to characterize the NTFET device. The device characteristic may be used later as a baseline for subsequent calibration of the device's electrical response.

After determining the device characteristic, a polymer functionalization layer may be deposited over the device at step 206. For example, the device may be submerged in a 10 wt % solution of poly(ethylene imine) (PEI, average molecular weight ˜25 000, Aldrich) and poly(ethylene glycol) (PEG, average molecular weight 10 000, Aldrich) in water overnight, followed by thorough rinsing with water. Commercial polyethyleneimine (PEI) may be used; this form is highly branched, has a molecular weight of about 25 000, and contains about 500 monomer residues. About 25% of the amino groups of PEI are primary with about 50% secondary, and 25% tertiary. After the coating process, a thin layer (for example, <10 nm) of polymer material should coat the devices. The finished polymer coating may be observed by atomic force microscopy.

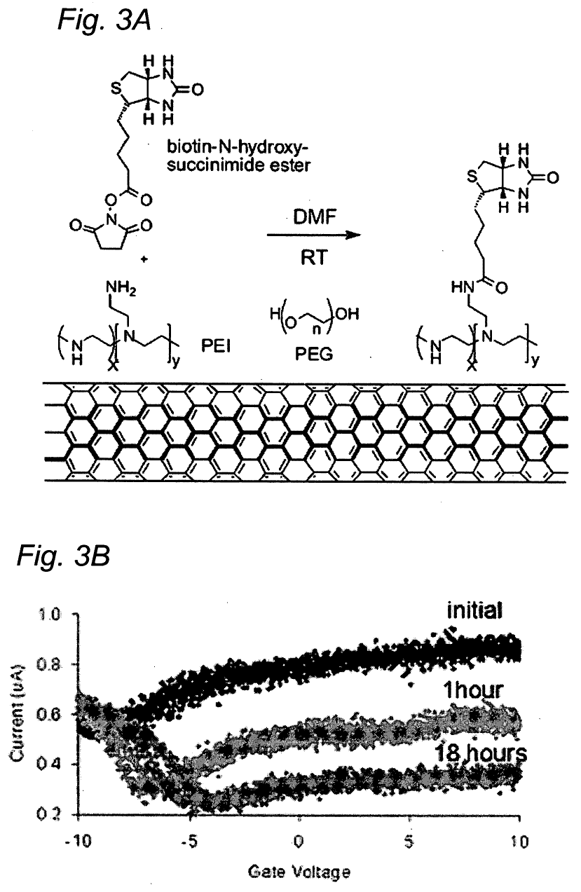

At step 208, the desired biomolecular receptor may be bonded to the polymer layer. If biotin is the desired receptor, a polymer-coated device may be biotinylated by submerging in a 15 mM DMF solution of biotin-N-hydroxysuccinimide ester (Sigma) at room temperature. This compound readily reacts with primary amines in PEI under ambient conditions, leading to changes of the device characteristic as will be discussed below. After soaking overnight, devices may be removed from solution, rinsed with DMF and deionized water, blown dry in nitrogen flow, and dried in a vacuum. FIG. 3A depicts a chemical scheme by which biotin may be attached to the polymer coating. FIG. 3B shows an exemplary transconductance curve for a PEI-coated device prior to the biotinylating reaction, and after 1 hour and 18 hours, respectively, of the reaction.

The device characteristics may be examined after drying, as reported herein. While the device may also exhibit a response in a buffer or other fluid, the examples herein should serve to illustrate the changes of the device characteristic, brought about by different chemical and biological modifications. Such direct correspondence may be somewhat obscured in a buffer environment.

Illustrative results are reported below. After drying, biotinylated polymer-coated devices constructed according to the foregoing description were exposed to a 2.5 μM solution of streptavidin 15 in 0.01 M phosphate buffered saline (pH ) 7.2, Sigma) at room temperature for 15 min. Subsequently, the devices were thoroughly rinsed with deionized water and blown dry with nitrogen.

An atomic force microscope (AFM) image of one of the devices after exposure to streptavidin labeled with gold nanoparticles indicated the presence of streptavidin. Based on the image, it appeared that streptavidin was effectively attached to the biotinylated PEI polymer coating the nanotubes. The imaged device comprised a nanotube about 800 nm long, and approximately 80 streptavidin molecules were surmised to be in direct interaction with the nanotube conducting channel.

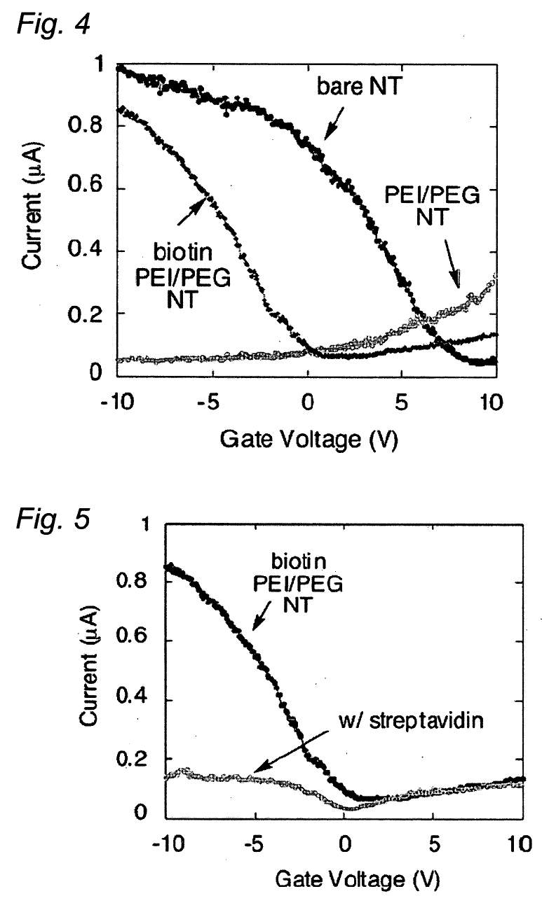

The device characteristic of the sensor before chemical modification was p-type in an ambient environment, presumably due to exposure to oxygen. Coating the device with the mixture of PEI and PEG polymers resulted in an n-type device characteristic, as shown by FIG. 4. The electronic characteristic of the device after 18 h of biotinylation reaction is also depicted in FIG. 4. Note that the p-type conductance observed before coating with PEI is not fully recovered after functionalization with biotin.

The effect of exposing the biotinylated polymer-coated device to a streptavidin solution and the control experiments (conducted on different devices) is shown in FIG. 5. A striking loss of source-drain current for negative gate voltages after exposure to streptavidin and consequent streptavidin-biotin binding is evident, with little shift of the device characteristic toward negative or positive gate voltage.

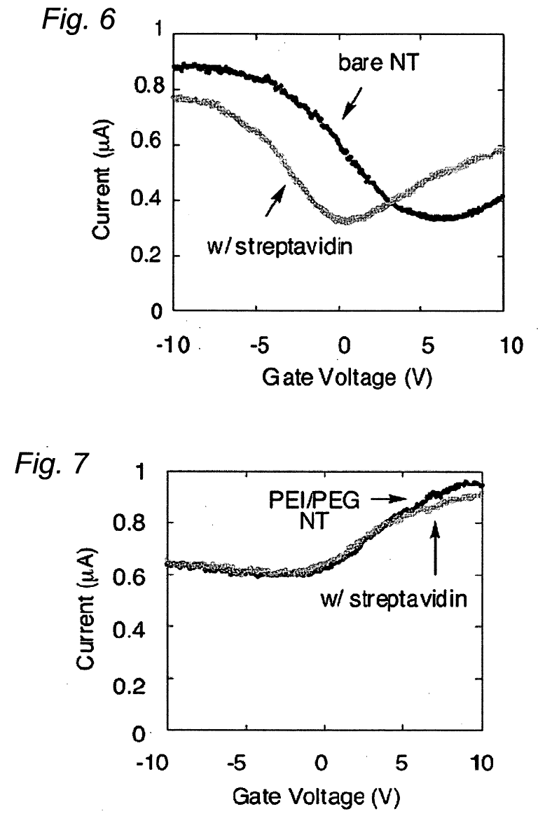

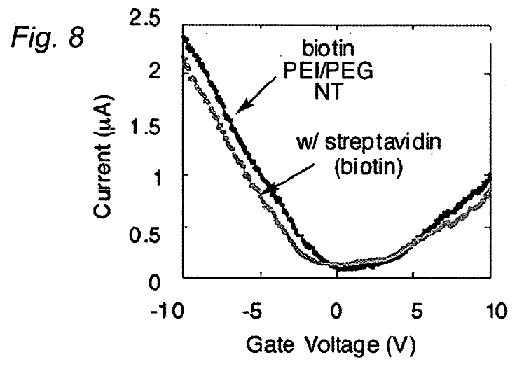

Several control experiments were performed to demonstrate the effectiveness of the device architecture in avoiding false positives and in detecting specific protein binding. First, the uncoated NTFET device was exposed to streptavidin. A change of the device characteristic, as shown in FIG. 6, may indicate attachment of streptavidin to the device. Note, however, that in this case the primary effect is the shift of the device characteristic toward negative gate voltage. In contrast, when the device was polymer-coated, but not biotinylated, no changes occurred upon exposure to streptavidin, as demonstrated by FIG. 7. This suggests the effectiveness of the polymer coating in preventing direct, nonspecific interaction of streptavidin with the nanotube. Finally, addition of a streptavidin in which the biotin-binding sites were blocked by complexation with excess biotin produced essentially no change in device characteristic of the biotinylated polymer-coated device, as demonstrated by FIG. 8.

Several conclusions on the effect of biomolecules on the device electronics may be drawn. First, exposing the bare, uncoated device to streptavidin leads to the shift of the transconductance toward negative gate voltages, thereby rendering the device less p-type, with little reduction in the magnitude of the transconductance. This indicates that the primary effect of the nanotube-streptavidin binding is a charge-transfer reaction with streptavidin donating electrons to the nanotube. Biotin-streptavidin binding has a different effect; in this case the current is reduced. At the same time the device characteristic is modified only for negative gate voltages as shown by FIG. 5, leaving the transcoductance in the positive gate voltage region unaffected.

Interestingly, similar effects may be observed in devices to which charge carriers were deposited. Such observed effects may be due to localization (delocalization) of positively (negatively) charged ionic entities by a negatively (positively) charged surface. Such a mechanism may also be effective with the disclosed nanotube device, and the mechanism may open the way for electronic modification of bioreactions.

With improvements in NTFET devices, they may also be rendered sensitive enough that single protein detection and monitoring can be achieved. As can be inferred from FIG. 5, the total change in transconductance exceeds the noise level by a factor of about 10. According to the AFM image of a device described above, there are approximately 100 protein molecules in close proximity to the carbon nanotube. Combining these two numbers, our current detection level is estimated to be of the order of 10 streptavidin molecules.

Similar detection sensitivity can be inferred from experiments we have conducted on uncoated nanotubes incubated with streptavidin, for which illustrative results are shown in FIG. 6. This is in contrast to the relatively modest change observed in devices where the active element is a nanowire—a channel with a substantially larger cross section.

Thus, label-free electronic sensing with a nanotube based transducer as the central sensor element may provide other significantly useful features in the detection of biological molecules. Such sensors are small, fast, require very little power, and thus generate little heat. The active sensing area is sized for individual proteins or viruses, and small sample volume in general, and is extremely sensitive as all the current passes through the detection point. Importantly, devices can be made specific to individual molecules, and potentially their response to different molecules can be controlled by using chemical and biological functionalization. Direct detection of specific oligonucleotides, in some ways, is typically even more challenging, and thus represents information more valuable than that of detecting individual proteins. Oligonucleotides in a sample generally show a high degree of variation, based on sequence, and often species of particular interest are rare from two perspectives, as a sample can contain populations of many oligonucleotide species very similar to the ones of interest, and at much higher concentrations.

The principles and practice of the invention may contribute, in due course, to the development of cell-based electronic sensing: measuring the electronic response of living systems, and to using nanoscale devices for in-vivo applications directed toward cellular physiology, medical screening, and diagnosis. Sensor devices may be constructed, according to the principles of the invention, wherein surface charges can be created on the sensing element when the biological molecules are immobilized, by applying a voltage between elements of the sensor. Such surface charges should interact with the charged bio-molecules, providing further opportunities for selective electronic detection of biomolecules, or electrical manipulation of biological reactions at a molecular level. Operation of a device according to the invention in this manner may therefore merit further study.

Having thus described a preferred embodiment of a nanotube sensor for selective sensing of biomolecules, and a method for constructing it, it should be apparent to those skilled in the art that certain advantages of the within system have been achieved. It should also be appreciated that various modifications, adaptations, and alternative embodiments thereof may be made within the scope and spirit of the present invention. For example, a biotin-streptavidin device has been illustrated, but it should be apparent that the inventive concepts described above would be equally applicable to devices that make use of other receptor/biomolecule combinations. The invention is further defined by the following claims.

What is claimed is:

1. A nanotube sensor, comprising:
a nanotube coated with a hydrophilic polymer; and
a molecular receptor compound bound to the polymer, the molecular receptor compound configured to bind a biological molecule.

2. The nanotube sensor of claim 1, wherein the biological molecule is a protein.

3. The nanotube sensor of claim 1, wherein the biological molecule is an oligonucleotide.

4. The nanotube sensor of claim 1, wherein the molecular receptor compound comprises a moiety of the biological molecule.

5. The nanotube sensor of claim 1, wherein the molecular receptor compound comprises biotin.

6. The nanotube sensor of claim 1, further comprising a source electrode connected to the nanotube, and a drain electrode connected to the nanotube.

7. The nanotube sensor of claim 6, further comprising a plurality of nanotubes connected to the source electrode, and to the drain electrode.

8. A method for fabricating a nanotube biosensor, the method comprising:
fabricating a NTFET;
coating a nanotube of the NTFET with a hydrophilic polymer layer; and
attaching a bioreceptor compound to the hydrophilic polymer layer.

9. The method of claim 8, further comprising determining a device characteristic of the NTFET after the fabricating step.

10. The method of claim 8, wherein the fabricating step further comprises constructing a nanotube spanning opposing electrodes over a substrate.

11. The method of claim 8, wherein the coating step further comprises a process selected from: immersing the NTFET in a polymer solution, spincasting a polymer solution onto the NTFET, or dropcasting a polymer solution onto the NTFET.

12. The method of claim 8, wherein the attaching step further comprises covalently bonding the bioreceptor compound to the hydrophilic polymer layer.

13. The method of claim 8, wherein the attaching step further comprises bonding molecules of the bioreceptor compound to amine groups of the hydrophilic polymer layer.

14. A method for detecting a biological compound using a sensor comprising a nanostructure device coated with a hydrophilic polymer bound to an electron-donating compound configured to bind to the biological compound, the method comprising:
exposing the nanostructure device to a sample; and
signaling detection of the biological molecule based on a value of transconductance across the nanostructure device.

15. The method of claim 14, wherein. the signaling step further comprises signaling a change in a device characteristic.
